(19) United States
(12) Patent Application Publication
(10) Pub. No.: US 2016/0313420 A1
(43) Pub. Date: Oct. 27, 2016

(54) UNILATERAL MAGNETIC RESONANCE SCANNING DEVICE FOR MEDICAL DIAGNOSTICS

(71) Applicant: SIRONA DENTAL SYSTEMS GMBH, Bensheim (DE)

(72) Inventors: Volker RASCHE, Erbach (DE); Erich HELL, Illingen (DE); Johannes ULRICI, Darmstadt (DE)

(73) Assignee: DENTSPLY SIRONA Inc., York, PA (US)

(21) Appl. No.: 15/105,108

(22) PCT Filed: Dec. 18, 2014
(86) PCT No.: PCT/EP2014/078395

§ 371 Date: Jun. 16, 2016

(30) Foreign Application Priority Data
Dec. 19, 2013 (DE) 10 2013 226 745.2

(51) Int. Cl. G01R 33/3815; G01R 33/341; A61B 5/055; G01R 33/383; G01R 33/381; G01R 33/38; G01R 33/28

(52) U.S. Cl. CPC G01R 33/3815; G01R 33/3808; G01R 33/3804; G01R 33/341; G01R 33/285; G01R 33/383; G01R 33/381; A61B 5/0555

(57) ABSTRACT

Disclosed is a scanning device for magnetic resonance imaging for medical diagnostics, more particularly for dental-medical diagnostics or ENT diagnostics, having a main magnet for generating a static main magnetic field having a homogeneous region, and having at least one transmitting and/or receiving coil for emitting and/or receiving a radio-frequency magnetic field. Provision is made, in particular, for the main magnet to be formed by two poles of magnetically opposite polarities at the end side, such that the static main magnetic field generated by the two poles at the end sides thereof, including the homogeneous region, projects beyond the end sides of the poles.

### TECHNICAL FIELD

Disclosed herein is a scanning device for magnetic resonance imaging for medical diagnostics, more particularly for dental-medical diagnostics or ENT diagnostics, comprising a main magnet for generating a static main magnetic field, and comprising at least one transmitting and/or receiving coil for emitting and/or receiving a high-frequency magnetic field.

### BACKGROUND

The invention is based on a scanning device for magnetic resonance imaging, and the use of such a device in medical diagnostics of the kind in the independent claims.

Magnetic resonance imaging (MRI) makes it possible to generate very highly detailed images of organs and tissues of the human or animal body, or a section thereof. Such images are otherwise only feasible by means of x-ray methods or other ionizing radiation that involve the known effects harmful to health.

In MRI, a static magnetic field with a high field strength for penetrating the tissue or organ is generated in a known manner. This magnetic field causes the numerous protons in the tissue to align. Consequently, one of the factors which determines the resolution and quality of the generated images is the homogeneity of the static magnetic field in the scanned region.

This magnetic alignment is disturbed by a high-frequency magnetic field which overlaps the static main magnetic field and is emitted into the tissue by a transmitting coil such that the protons emit a signal in a known manner that is detectable by receiving coil.

By means of an additional irradiating magnetic gradient field, the protons are caused to perform site-dependent precession movements in the direction of the gradient at different speeds, which makes it possible to extract spatial information from the detected signals by means of Fourier analysis. Two-dimensional or three-dimensional images can be generated by using such gradients in different directions in space.

The MRI method yields particularly contrast-rich images for distinguishing between different soft tissues, in particular for distinguishing between healthy and diseased tissue.

With known clinical MRI systems based on a permanent magnet technology or a magnet technology founded on superconductive magnets, the body of the patient to be examined is at least partially surrounded by the main magnet generating said static field. In the example of a magnetic dipole, the main magnet consists of two pole shoes that are located on opposite sides of the body and are connected to each other by an iron yoke.

A disadvantage of such systems is that the homogeneous region of the static magnetic field which is relevant for imaging runs through the entire body to be scanned (see FIG. 1) even when only a small region is of interest. The amount of magnetic material used and the system dimensions are larger than necessary, which leads to high costs. Since all of the locations in the homogeneous region of the static magnetic field contribute to the scanning or measuring signal, the signal/noise ratio is worse than is the case with a more spatially limited homogeneous magnetic region. Another disadvantage is the effort involved in positioning the patient in the main magnet.

As is the case with the known Halbach geometry of permanent magnet systems, or a cylindrical geometry of superconductive systems in which the main magnet encloses the patient, the homogeneous region of the static magnetic field of closed magnets is only in the center of the magnet, which makes it very difficult to position the patient.

The cited disadvantages can be overcome by unilaterally configured or unilaterally acting magnets. A corresponding unilateral magnet suitable for nuclear magnetic resonance measurements is known from U.S. Pat. No. 6,489,872 B1. With the magnet arrangements disclosed therein, the same magnetic poles are opposite each other in a lateral (side) direction. This either reduces the homogeneous field region suitable for magnetic resonance measurements to a relatively small volume (the so-called sweet spot), or the possible width of the field outside of the magnet is restricted to relatively low values. Furthermore, most of the arrangements of the magnets disclosed therein are relatively complex or technically involved to produce or operate.

A unilateral magnetic resonance sensor is also disclosed in DE 20 2006 002 074 U1. The sensor has four permanent magnets that are separated by two rectangular gaps which serve to generate a static magnetic field. A magnetic field with sufficient homogeneity can only be generated by the arrangement of four permanent magnets. This sensor is both difficult to produce and to operate due to the four permanent magnets; in addition, it has a relatively large volume and weight and is therefore not suitable for the field of dental-medical diagnostics.

For the aforementioned reasons, the cited MRI systems are in particular unsuitable for dental-medical diagnostics or examinations.

### SUMMARY

The present disclosure is based on the concept of providing an MRI scanning device in which the aforementioned main magnet is not arranged around the patient, but rather can approach the patient, or tissue or organ of the patient to be examined, unilaterally and hence unhindered from the outside. The underlying diagnostics of the tissue or organ of the patient to be examined is preferably dental diagnostics or ears, nose and throat (ENT) diagnostics.

According to the present disclosure, the approachability is enabled in that the main magnet is formed from at least two, preferably two unilaterally arranged poles with differing magnetic polarity directed to the outside so that the magnetic fields generated by the at least two poles run outside of the region enclosed by the poles of the main magnet (i.e., unilaterally), and the main magnet can therefore approach the patient from one side without being restricted by the magnetic poles. The magnet can have the shape of a horseshoe magnet. Of course, the main magnet can also be formed by a multi-pole magnet, such as quadrupole magnet, etc.

The present disclosure is based on the concept that, despite the unilateral arrangement of the least two poles, a static magnetic field with sufficient homogeneity can be unexpectedly generated.

It should be noted that the unilateral static magnetic field can be generated both by an arrangement of magnets according to this disclosure as well as by the corresponding arrangement of pole shoes. With regard to an arrangement of magnets, the main magnet can be formed by two magnet blocks having opposite polarity arranged on an iron yoke or iron core. With regard to a superconductive magnet technology, superconductive coils can be correspondingly arranged.

The cited pole shoes of the main magnet are known components made of a material with a high magnetic permeability such as iron which, in the present case, can serve to capture and bundle the magnetic fields or field lines generated by the permanent magnet at the rear of the magnet sensor and released into free space at that location in order to thereby minimize magnetic loss.

The pole shoes can be implemented in different arrangements, for example by at least two magnets with an external square or rectangular shape, or by two adjacent semicircular or annular magnets formed as half cylinders in which an opening is cut out in the middle. Especially due to this opening, the field lines extend far enough into space that they can penetrate the tissue or organ to be examined with sufficient depth.

Between the at least two poles, a cutout can be arranged which can be used to position a patient. Accordingly for example, the head of the patient can be positioned in the cutout so that the head is at least partially surrounded by the at least two poles.

The scanning device as disclosed herein can be realized both with permanent magnets as well as with electromagnets or superconducting magnets and can preferably be used in dental-medical diagnostics. In addition, use in general medical diagnostics is possible, in particular for diagnosis of tissues or organs close to the surface of the body. In addition, the device can be used in the field of computer tomographic imaging where the individual photographic slices are generated by a relative movement between the scanning device and patient, or by a corresponding variation of the cited magnetic gradient field.

Additional advantages and features are found in the following description of preferred exemplary embodiments in conjunction with the drawings. The individual features can be realized by themselves or in combination with each other. In the drawings, identical or functionally corresponding features are provided with the same reference numbers.

### BRIEF DESCRIPTION OF THE DRAWINGS

In the drawings:

FIG. 1 shows a schematic side view of an MRI scanning device according to the prior art.

FIGS. 2a, b show schematic views of two exemplary embodiments of an MRI scanning device according to the disclosure;

FIG. 3 shows a scanning device according to the disclosure that is positioned by means of a holding device in the oral region of a sitting or standing patient for performing dental-medical or ENT medical diagnostics;

FIG. 4 shows a scanning device described herein that is positioned in the head region of a recumbent patient for performing a dental-medical diagnosis;

FIGS. 5a, b show two exemplary embodiments of a main magnet according to the disclosure, each with an integrated gradient coil;

FIG. 6 shows a scanning device according to the disclosure held by a non-adjustable holding device, wherein a patient can be positioned on a laterally and height-adjustable chair sitting on a scanning device;

FIGS. 7a-c show three exemplary embodiments of a holding device for securing a patient's head to a scanning device according to the present disclosure, and

FIGS. 8a, b show two exemplary embodiments of an arrangement of a transmitting/receiving device on a scanning device according to the disclosure.

### DETAILED DESCRIPTION

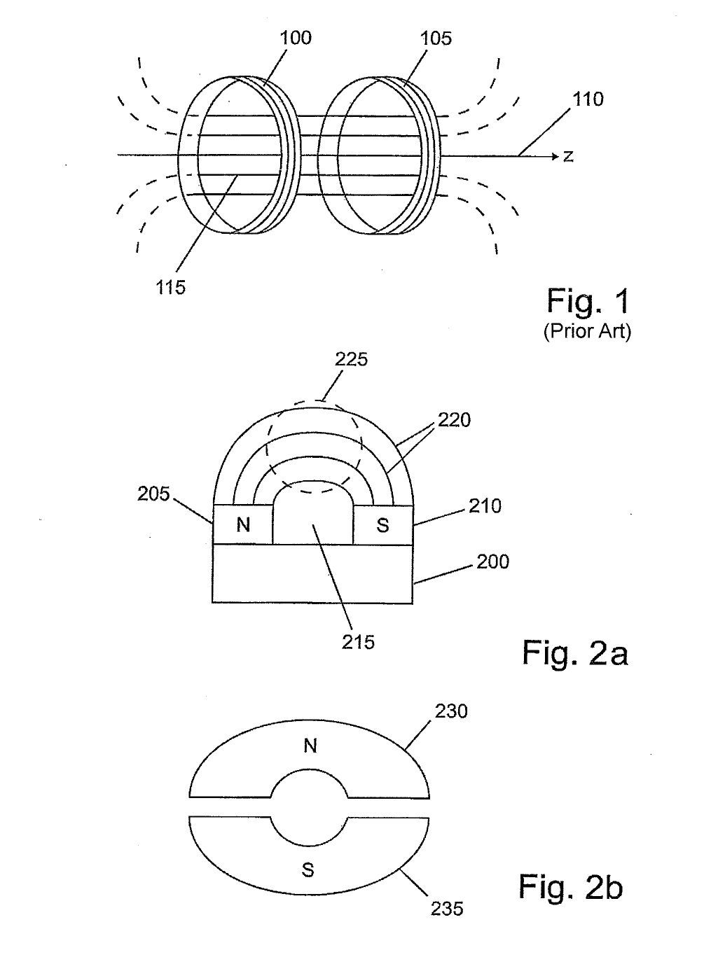

FIG. 1 shows a known arrangement consisting of main magnets, formed from two pole shoes 100, 105, of an MRI system. The main magnet 100, 105 encloses the patient to be examined who is positioned in this depiction in the z-direction 110 of the two pole shoes 100, 105 as is conventional with whole body systems. Alternately, especially in the case of an open magnet system, the patient can also be positioned between the coils of the main magnets 100, 105, i.e., orthogonal to the z-direction 110. A static magnetic field 115 generated by the main magnet 100, 105 therefore mainly passes through the body of the patient, which makes locally limited examinations such as in the head or oral region difficult or impossible. Due to the high cost of purchasing and operating such a magnetic system, this MRI system is unsuitable, especially for dental diagnostics.

FIG. 2a shows an exemplary embodiment of a main magnet according to the present disclosure in a schematic side view. In the present case, the main magnet is formed from two magnet blocks 205, 210 which are arranged with opposing polarity on an iron yoke 200. A cutout 215 is formed between the two magnet blocks 205, 210. The lateral extension of the cutout essentially corresponds to the lateral extension of the magnet blocks 205, 210. Instead of the iron yoke 200, the main magnet can also be formed from a continuous horseshoe magnet.

Due to the opposing polarity resulting at the front side, the resulting magnetic field (shown in FIG. 2a) has magnetic field lines 220 that are formed in a particularly homogeneous manner in a region 225 above the cutout 215. The spatial extension of this homogeneous region 225 is at least 5 cm, both in a vertical and horizontal (lateral) direction. Within the homogeneous region, the static magnetic field has a magnetic field strength variation less than 50 ppm, and preferably less than 10 ppm.

It should be noted that the two poles can also be formed by two pole shoes arranged on the face of a permanent magnet.

The exemplary embodiment according to FIG. 2b shows the two poles 230, 235 of a main magnet according to the present disclosure in a front view. In this example, the poles are formed by two half-shell magnet blocks 230, 235 which are arranged so that together they form a nearly complete ring. The resulting static magnetic field possesses circular symmetry in the depicted plane. Correspondingly, the homogeneous region is formed as a circular disk.

Depending on the spatial extension of the tissue or organ to be examined, one or the other of the two exemplary embodiments may be particularly suitable or advantageous due to the spatial correspondence with the homogeneous magnetic field region.

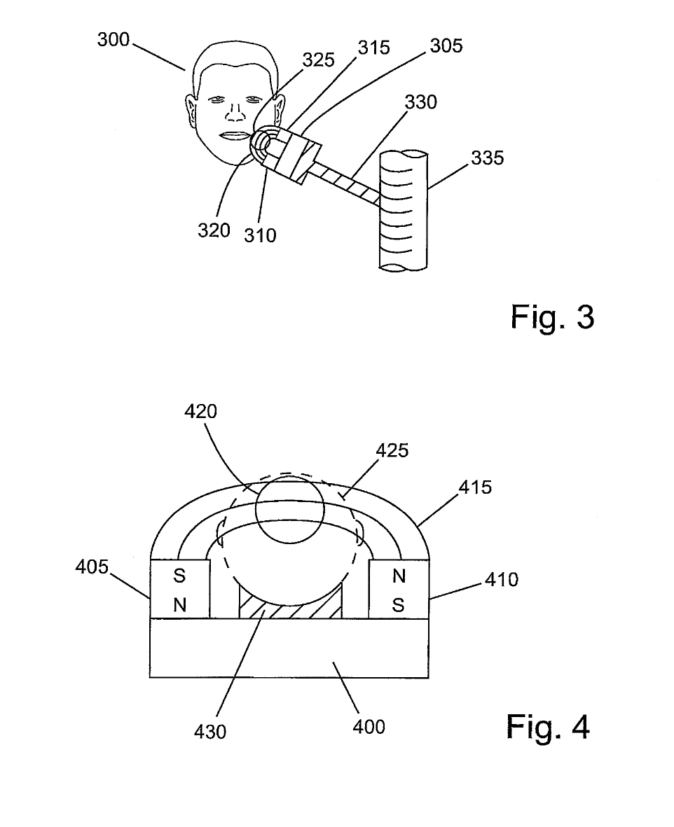

As can be seen from FIG. 3, the main magnet or permanent magnet 305-315 arranged on one side or laterally in a dental MRI system according to the present disclosure is positioned outside the patient 300 such that the homogeneous field region 325 of the static magnetic field 320 is located in the dental tissue to be examined, or teeth to be examined. In this exemplary embodiment, the patient 300 sits or stands, wherein the patient can be secured as close as possible to the scanning device, for example by means of a belt surrounding the head of the patient, or by means of a system consisting of inflatable pillows. The main magnet 305-315 is fastened to a holding device 330, 335, which in the present exemplary embodiment is formed by a cross-connection 330 and a column 335 connected to the cross connection 330 and to the floor (not shown).

In the exemplary embodiment shown in FIG. 4, the main magnet 400-410 is formed by two permanent magnet blocks 405, 410 arranged on a relatively wide iron yoke or iron core 400. Of course, instead of the iron core a corresponding arrangement with a horseshoe magnet can be provided. In this exemplary embodiment, the head 425 of the patient lies at least on the iron yoke 400, or on the bottom part of the magnet in the case of a horseshoe magnet. The head 425 is securely positioned by a pillow 430. In the present exemplary embodiment, the head 425 lies in the above-described opening or cutout in the main magnet 400-410. The generated static main magnetic field 415, in particular the homogeneous region 420, floods the front head region of the patient 425.

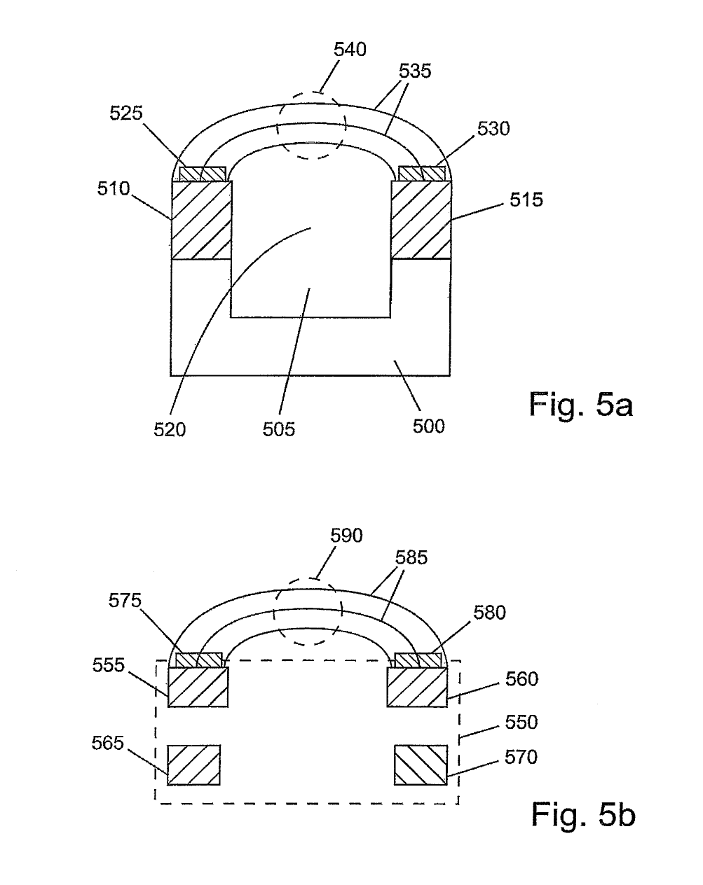

In the exemplary embodiment shown in FIG. 5a, two permanent magnet blocks 510, 515 are arranged on an iron yoke 500. The iron yoke 500 has a cutout 505 that, in the example, has a somewhat greater lateral extension than one of the two permanent magnet blocks 510. In the present case, the resulting static main magnetic field 535 as well as the homogeneous region 540 are also sketched. Of course, the two permanent magnet blocks 510, 515 can possess the same lateral extension as the corresponding top sides of the iron yoke 500, whereby the transitions between the magnets and iron yoke 500 can also be configured or arranged flush (that is, without the projections shown in FIG. 5a).

In addition to the main magnets 500, 510, 515, gradient coils 525, 530 are arranged on the permanent magnet blocks 510, 515 by means of which a magnetic gradient field (not shown) can be generated that overlaps the main magnetic field 535. The particular advantage of such a gradient field in dental-medical diagnostics is the possibility of a three-dimensional representation of the entire masticatory apparatus or individual teeth, which can significantly improve the quality of the diagnosis.

It should be noted that the gradient coils 525, 530 and/or transmitting/receiving coil(s) (not shown) can also be arranged in the region of the cutout 520 provided between the two permanent magnet blocks 510, 515. Furthermore, transmitting and receiving coil(s) can be formed by a single coil.

In addition to the above-described exemplary embodiments, temperature stabilization can be provided. Temperature stabilization can be provided, for example, by water cooling, wherein the flow of cooling water is regulated by the temperature measured at the magnet. Alternately, the system can also be heated by an electric heater, the heating being controlled with reference to a measured temperature.

In the exemplary embodiment shown in FIG. 5b, a superconductive magnet can be used instead of a conventional permanent magnet. Superconductive coils 555, 560 for generating the main magnetic field 585 with a homogeneous region 590 are arranged in a conventional housing or carrier 550. In addition, shielding coils 565, 570 are arranged to the rear in relation to the superconductive coils 550, 560 to actively shield the generated main magnetic field 585 to the rear. Gradient coils 575, 580 are also additionally provided in this exemplary embodiment. More than one coil per magnetic pole can also be arranged to improve the homogeneity of the magnetic field.

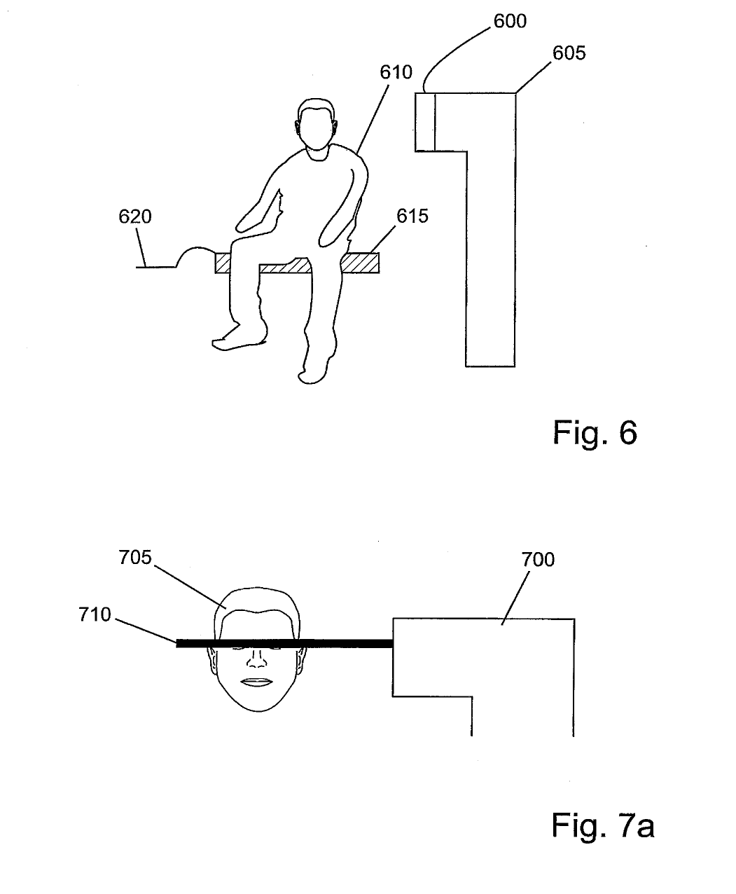

Given the very small size of the scanning device according to the disclosure herein, it can be arranged at the head side 600 of a column 605 as illustrated in FIG. 6. In the present example, the patient 610 sits on a height-adjustable seat or chair 615 that can be attached to a room wall, for example, by an articulated joint 620. Furthermore, the seat can be designed to rotate in the seat plane to improve the positioning of the patient 610 for the examination. The patient can be brought to the scanning device 600 by means of the height-adjustable seat 615.

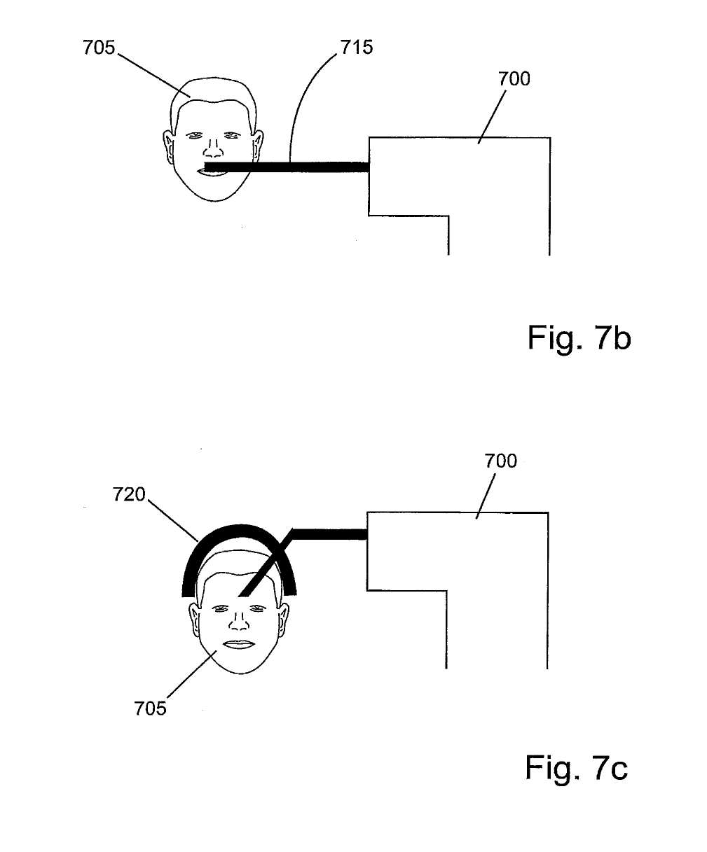

FIGS. 7a, 7b and 7c schematically portray three different exemplary embodiments for fixing the head of the patient 705 relative to the scanning device. The scanning device is portrayed as an integral part of a holding device 700 of a relevant MRI system. In FIG. 7a, the scanning device is secured by means of a flexible strap 710, such as a rubber strap, to the scanning device. In FIG. 7b, securing is effected by an occlusal splint 715, which is customary in dentistry, whereas the entire head of the patient 705 is secured by a headrest in FIG. 7c.

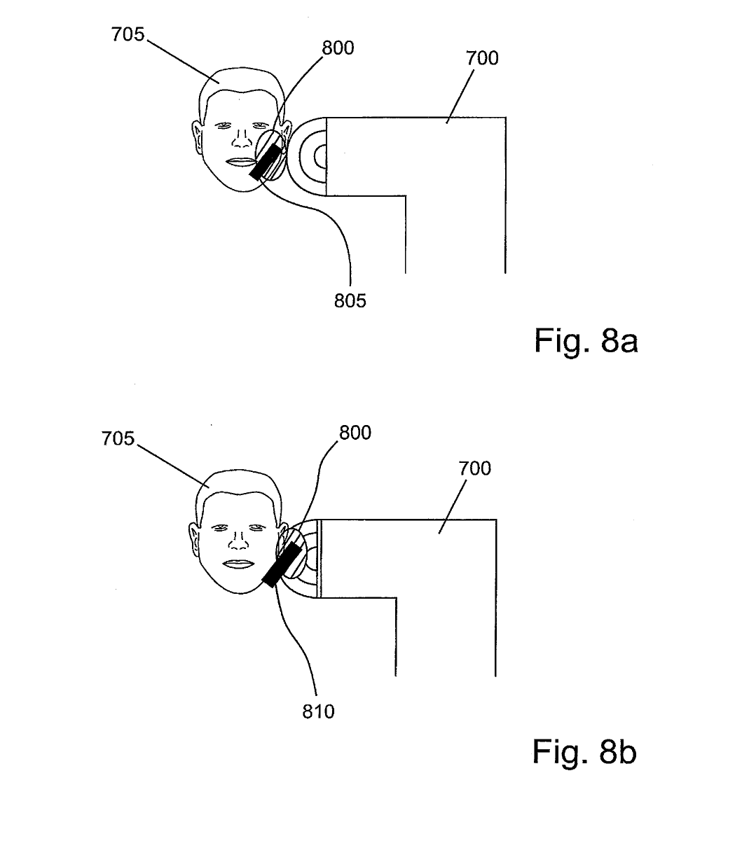

FIGS. 8a and 8b show two exemplary embodiments to illustrate the different positions in which one or more of the cited transmitting/receiving coils can be arranged relative to the main magnetic field, or the homogeneous region 800 of the main magnetic field. In the first example according to FIG. 8a, the transmitting/receiving coil 805 is located in the oral cavity of the patient 705. The advantage of this arrangement is that, for example, structures of teeth can be imaged with a higher resolution. In the second exemplary embodiment according to 8b, the transmitting/receiving coil 810 is arranged outside of the oral cavity of the patient 705, thereby enabling comprehensive imaging, for example of the entire oral cavity.

1. A scanning device for magnetic resonance imaging for medical diagnostics, comprising a main magnet for generating a static main magnetic field having a homogeneous region, at least one transmitting and/or receiving coil for emitting and/or receiving a high-frequency magnetic field, wherein the main magnet is formed by at least two poles of magnetically opposite polarities at the face, wherein the static main magnetic field generated by the two poles at the faces thereof, including the homogeneous region, projects beyond the faces of the poles.

2. The device according to claim 1, wherein a cutout is arranged between the at least two poles.

3. The device according to claim 2, wherein the cutout is used for patient positioning.

4. The device according to claim 3, wherein the body part of the patient to be diagnosed is positioned in the cutout such that the body part is surrounded at least partially by the at least two poles.

5. The device according to any of the preceding claims, wherein at least one gradient coil is arranged on the main magnet to generate a magnetic gradient field overlapping the main magnet.

6. The device according to claim 1, wherein the static main magnetic field, including the homogeneous region, extends beyond the faces of the at least two poles by at least 5 cm.

7. The device according to claim 1, wherein the at least two poles are formed by pole shoes arranged on the face of permanent magnets, or by poles of superconductive coils, or by electromagnets.

8. The device according to claim 1, wherein the static main magnetic field in the homogeneous region is constant, with a variability of less than 50 ppm, and preferably less than 10 ppm.

9. The device according to claim 1, wherein the homogeneous region of the static main magnetic field comprises at least a volume with a spatial extension of at least 5 cm.

10. The device according to claim 1, wherein the lateral extension of the cutout substantially corresponds to the lateral extension of the at least two poles.

11. The device according to claim 1, wherein the main magnet has at least one permanent magnet or at least one superconductive magnet.

12. The device according to claim 1, wherein the at least one gradient coil is arranged on the face of the main magnet, or in the region of the cutout provided between the at least two poles.

13. The device according to claim 1, wherein the at least one transmitting and/or receiving coil is arranged on the main magnet or in the region of the cutout provided between the at least two poles.

14. The device according to claim 1, wherein the transmitting coil and the receiving coil of the at least one transmitting and receiving coil are formed by a single coil.

15. The device according to claim 1, wherein the at least one transmitting and receiving coil is designed to be positionable on the patient or within the oral cavity of the patient.

16. The device according to claim 1, wherein the main magnet is held by a holding device that can be moved in at least one direction.

17. The device according to claim 1, wherein a positioning device is arranged for a patient that can be moved in least one direction with the patient.

18. The device according to claim 1, wherein means are provided to stabilize temperature.

19. The device according to claim 8, wherein the variability is less than 10 ppm.
